(19) United States
(12) Patent Application Publication
(10) Pub. No.: US 2019/0214109 A1
(43) Pub. Date: Jul. 11, 2019

(54) Methods For Finding Genome Rearrangments From Sequencing Data

(71) Applicant: Rutgers, The State University of New Jersey, New Brunswick, NJ (US)

(72) Inventors: Andrey Grigoriev, Medford, NJ (US); Sean Douglas Smith, Woodstown, NJ (US)

(21) Appl. No.: 16/241,725

(22) Filed: Jan. 7, 2019

Related U.S. Application Data
(60) Provisional application No. 62/614,828, filed on Jan. 8, 2018.

(51) Int. Cl. G16B 20/20; G16B 30/10; G16B 15/10; C12Q 1/68

(52) U.S. Cl. CPC G16B 20/20; C12Q 1/68; G16B 15/10; G16B 30/10

(57) ABSTRACT

The present disclosure generally relates to finding genome rearrangements from sequencing data. DNA sequence analysis systems and methods directed to identifying all sequence variants in a genome are described herein. Such systems and methods demonstrate distinct and improved features relating to the accuracy and speed with which all sequence variants in a genome are identified.

### RELATED APPLICATIONS

This application claims priority of U.S. Provisional Application No. 62/614,828, filed Jan. 8, 2018, the entirety of which is incorporated herein by reference for all purposes.

### FIELD OF INVENTION

The present disclosure generally relates to finding genome rearrangements from sequencing data.

### BACKGROUND

Due to the dropping costs of sequencing there is a large increase in population sequencing studies, ranging from just a few individuals to several thousands of genomes. While these studies typically report single-nucleotide variants (SNVs), other types of variants such as short insertions/deletions (indels) and larger structural variants are seldom analyzed. There are currently several main impediments to analyses of such other types of variants, such as: (i) lack of best practices in structural variant (SV) detection often leads to employing several variant finders thereby producing divergent sets of predictions, (ii) partly because of (i), current variant finding pipelines are slow and take a long time to run on single or multiple samples, and (iii) none of such current variant finding pipelines combines all available evidence from single or multiple samples for detecting all variant types.

### SUMMARY

In some embodiments, the present disclosure describes a computer based process for genome sequencing. In some embodiments, the present disclosure describes an integral computational platform for fast, accurate detection of genome variants from next-generation sequencing (NGS) data for comparative genomics. Next-generation sequencing refers to non-Sanger-based high-throughput DNA sequencing technologies. Millions or billions of DNA strands can be sequenced in parallel, yielding substantially more throughput and minimizing the need for the fragment-cloning methods that are often used in Sanger sequencing of genomes.

In some embodiments, the present invention may be utilized for healthcare (e.g. diagnostics, stratified drug trials, personalized medicine), agriculture (e.g. marker or variant assisted breeding), and research. In some embodiments for example, the present invention may be utilized in genome based diagnostic tests for diseases (e.g. analysis of predisposition or presence of a variant in a disease fluid or tissue sample) and patient cohort analysis (e.g. presence of variants for patient stratification for clinical trials).

Current tools for analyzing next-generation sequencing (NGS) data and identifying structural variants (SVs) only find subsets of variants. In some embodiments, unlike currently available variant detection tools, embodiments of the present disclosure can detect all types of variants including but not limited to: single-nucleotide variants (SNVs), short insertions/deletions (Indels), and structural variants (SVs) such as deletions, duplications inversions, and translocations.

In an aspect, a DNA sequence analysis system is presented, comprising:
computing module, configured to:
   receive DNA sequencing data;
      wherein the DNA sequencing data is a plurality of
         non-paired sequenced reads, or
         paired sequenced reads with unsequenced DNA between them,
      of at least one genome of a subject;
   receive at least one DNA reference sequence and reference DNA alignment data for the at least one DNA reference sequence;
   analyze the reference DNA alignment data and the at least one DNA reference sequence to obtain a plurality of distinct reference mismatch identifying data type outputs, for non-paired reads comprising:
      i) an abnormal read depth identifying data type output,
      ii) a single nucleotide variant identifying data type output,
      iii) a short insertion/deletion (indel) identifying data type output, or
      iv) a split-read mapping identifying data type output
   and, for paired reads, additionally comprising:
      v) a discordant mate identifying data type output,
      vi) an unmapped mate identifying data type output, or
      vii) a discordant read orientation identifying data type output;
   evaluate each respective genome position of the at least one genome of the subject using a joint analysis of all distinct data type outputs of the plurality of reference mismatch identifying data type outputs to identify all subject-specific genome variants corresponding to at least one genome variant type of a plurality of genome variant types;
   wherein each potential reference genome variant relative to the at least one reference DNA sequence is at least one of:
      a) a single-nucleotide variant,
      b) a short indel,
      c) a deletion,
      d) an insertion of a non-reference DNA sequence,
      e) an inversion,
      f) a duplication,
      g) a translocation between separate contiguous DNA stretches,
      h) a change in a copy number of parental alleles;
   wherein a speed of jointly identifying all genome variants of the plurality of genome variant types by jointly considering all distinct data type outputs of the plurality of reference mismatch identifying data type outputs is at least 1.5 fold higher than a speed of obtaining the same genome variants of the plurality of genome variant types, by separately identifying and then combining:
      i) one or more genome variants of each respective genome variant type of the plurality of genome variant types, or
      ii) one or more genome variants of each subset of respective genome variant types of the plurality of genome variant types;
   wherein an accuracy of jointly identifying all genome variants of the plurality of genome variant types by jointly considering all distinct data type outputs of the plurality of reference mismatch identifying data type outputs is equal to or higher than an accuracy of separately identifying the same all genome variants of the plurality of genome variant types, by separately identifying:
      i) all genome variants of each respective genome variant type of the plurality of genome variant types, or
      ii) all genome variants of each subset of respective genome variant types of the plurality of genome variant types.

In other words, both accuracy and speed will be improved by jointly considering all distinct data type outputs of the plurality of reference mismatches identifying data type outputs compared to identifying incomplete sets of variant types and combining the results of same.

As described herein, the DNA sequence analysis system utilizes GROM, which exhibits the ability to predict all variant types. GROM is, therefore, superior to other methods for detecting variants which are limited to only predicting particular types or groups of types of variants. Accordingly, as demonstrated by results presented herein, implementation of GROM in a DNA sequence analysis system and methods for detecting variants improves both accuracy and the speed with which DNA variants can be identified by the DNA sequence analysis system and methods described herein.

In a particular embodiment of the DNA sequence analysis system, the particular subject-specific genome variant is associated with a particular disease or a particular disorder. In a more particular embodiment, the particular disease or the particular disorder is a cancer.

In a still more particular embodiment, the particular subject-specific genome variant associated with the particular disease or the particular disorder corresponds to at least one abnormal genotype difference in at least one diseased body part of the subject from a non-diseased body part of the subject; and
further comprises:
identifying the at least one abnormal genotype difference, by jointly comparing each subject-specific genome variant identified in a first genome of the at least one diseased body part of the subject to each subject-specific genome variant identified in a second genome of the non-diseased body part of the subject.

In another particular embodiment of the DNA sequence analysis system, wherein, as part of the evaluation of each respective genome position of the at least one genome of the subject using a joint analysis of all distinct data type outputs of the plurality of reference mismatch identifying data type outputs, the computing module is further configured to:
produce during the evaluation at least one breakpoint cluster of reads supporting the same variant type,
   wherein a breakpoint cluster at a specific reference genome position is a set of reads or unsequenced DNA between paired reads supporting a breakpoint at that location for a specific variant type of a length approximation compatible with reference mismatch identifying data type outputs obtained from said reads;
identify at least one variant from the plurality of breakpoint cluster of reads by using common statistical evaluation for different variant types,
   wherein the identified presence of one variant type affects the evaluation of another variant type.

In some embodiments, the present disclosure utilizes the findings of one type of variant to further inform the computational protocol about possible effects on finding other types of variants. In some embodiments for example, the present disclosure factors detected changes in single-nucleotide variant (SNV) allele frequency as indicators of a possible structural variant (SV). In some embodiments for example, a detected structural variant (SV) affects in its vicinity, the parameters used for single-nucleotide variant (SNV) detection.

In a particular embodiment thereof, an identified heterozygous deletion could, for example, make heterozygous substitutions in the same region appear homozygous.

In another particular embodiment of the DNA sequence analysis system, wherein, as part of the evaluation of each respective genome position of the at least one genome of the subject using a joint analysis of all distinct data type outputs of the plurality of reference mismatch identifying data type outputs, the computing module is further configured to apply during the evaluation a nucleotide content weighting method for each genome position.

In another particular embodiment of the DNA sequence analysis system, wherein, as part of the evaluation of each respective genome position of the at least one genome of the subject using a joint analysis of all distinct data type outputs of the plurality of reference mismatch identifying data type outputs, the computing module is further configured to apply during the evaluation a nucleotide content bias normalization for each genome position.

In another particular embodiment of the DNA sequence analysis system, wherein, as part of the evaluation of each respective genome position of the at least one genome of the subject using a joint analysis of all distinct data type outputs of the plurality of reference mismatch identifying data type outputs, the computing module is further configured to apply during the evaluation a dinucleotide repeat bias normalization for each genome position.

In another particular embodiment of the DNA sequence analysis system, wherein, as part of the evaluation of each respective genome position of the at least one genome of the subject using a joint analysis of all distinct data type outputs of the plurality of reference mismatch identifying data type outputs, the computing module is further configured to:
utilize during the evaluation at least one sequence window with independently sliding borders for finding copy number changes based on read depth, and
to add at least one window with the copy number change borders to the breakpoint clusters supporting deletion and duplication type variants.

In another aspect, method is presented, comprising:
receiving, by a computing module, DNA sequencing data;
   wherein the DNA sequencing data is a plurality of
      non-paired sequenced reads, or
      paired sequenced reads with unsequenced DNA between them, of at least one genome of a subject;
receiving, by the computing module, at least one DNA reference sequence and reference DNA alignment data for the at least one DNA reference sequence;
analyzing, by computing module, the reference DNA alignment data and the at least one DNA reference sequence to obtain a plurality of distinct reference mismatches;
identifying, by computing module, data type outputs, for non-paired reads comprising:
   i) an abnormal read depth identifying data type output,
   ii) a single nucleotide variant identifying data type output,
   iii) a short insertion/deletion (indel) identifying data type output, or
   iv) a split-read mapping identifying data type output
and, for paired reads, additionally comprising:
   v) a discordant mate identifying data type output,
   vi) an unmapped mate identifying data type output, or
   vii) a discordant read orientation identifying data type output;
evaluating, by computing module, each respective genome position of the at least one genome of the subject using a joint analysis of all distinct data type outputs of the plurality of reference mismatch identifying data type outputs to identify all subject-specific genome variants corresponding to at least one genome variant type of a plurality of genome variant types;
wherein each potential reference genome variant relative to the at least one reference DNA sequence is at least one of:
   a) a single-nucleotide variant,
   b) a short indel,
   c) a deletion,
   d) an insertion of a non-reference DNA sequence,
   e) an inversion,
   f) a duplication,
   g) a translocation between separate contiguous DNA stretches, or
   h) a change in a copy number of parental alleles;
wherein a speed of jointly identifying all genome variants of the plurality of genome variant types by jointly considering all distinct data type outputs of the plurality of reference mismatch identifying data type outputs is at least 1.5 fold higher than a speed of obtaining the same genome variants of the plurality of genome variant types, by separately identifying and then combining:
   i) one or more genome variants of each respective genome variant type of the plurality of genome variant types, or
   ii) one or more genome variants of each subset of respective genome variant types of the plurality of genome variant types;
wherein an accuracy of jointly identifying all genome variants of the plurality of genome variant types by jointly considering all distinct data type outputs of the plurality of reference mismatch identifying data type outputs is equal to or higher than an accuracy of separately identifying the same all genome variants of the plurality of genome variant types, by separately identifying:
   i) all genome variants of each respective genome variant type of the plurality of genome variant types, or
   ii) all genome variants of each subset of respective genome variant types of the plurality of genome variant types. In a more particular embodiment, the change in the normal copy number of parental alleles comprises loss of heterozygosity

In a particular embodiment of the above method, a particular genome variant is a particular validated genome variant associated with a particular disease or a particular disorder. In a more particular embodiment of the method, the particular disease or the particular disorder is a cancer or a similar condition, wherein a diseased part of a body has a genotype different by one or more breakpoints from a healthy part of the body.

In another particular embodiment of the above method, a particular genome variant is identified, for non-paired reads as comprising:
i) an abnormal read depth identifying data type output,
ii) a single nucleotide variant identifying data type output,
iii) a short insertion/deletion (indel) identifying data type output, or
iv) a split-read mapping identifying data type output
and, for paired reads, additionally comprising:
   v) a discordant mate identifying data type output,
   vi) an unmapped mate identifying data type output, or
   vii) a discordant read orientation identifying data type output.

In another particular embodiment of the above method, a particular genome variant is identified as one of:
a) a single-nucleotide variant (SNV),
b) a short indel (insertion or deletion<50 nucleotides in length) compared to reference DNA,
c) a deletion compared to reference DNA,
d) an insertion of non-reference DNA sequence,
e) an inversion compared to reference DNA,
f) a duplication compared to reference DNA,
g) a translocation between contiguous stretches of reference DNA, or
h) a change in the normal copy number of parental alleles. In a more particular embodiment, the change in the normal copy number of parental alleles comprises loss of heterozygosity.

In another particular embodiment of the above method, a particular genome variant is one of:
a) a single-nucleotide variant (SNV),
b) a short indel (insertion or deletion<50 nucleotides in length) compared to reference DNA,
c) a deletion compared to reference DNA,
d) an insertion of non-reference DNA sequence,
e) an inversion compared to reference DNA,
f) a duplication compared to reference DNA,
g) a translocation between contiguous stretches of reference DNA, or
h) a change in the normal copy number of parental alleles. In a more particular embodiment, the change in the normal copy number of parental alleles comprises loss of heterozygosity.

In another particular embodiment of the above method, the particular genome variant is associated with a cancer. More particularly, the particular genome variant associated with a cancer is listed in Table 3.

In another particular embodiment of the above method, the method further comprises
(a) determining, by computer module, if a genome of the subject comprises the particular validated genome variant associated with the cancer,
   wherein identifying that the genome of the subject comprises the particular validated genome variant associated with the cancer selects the subject for at least one of a monitoring method or a diagnostic method relating to monitoring or diagnosing the cancer; and
(b) performing the at least one of the monitoring method or the diagnostic method relating to monitoring or diagnosing the cancer in the subject identified as having the genome comprising the particular validated genome variant associated with the cancer. In a more particular embodiment of the method, the monitoring method or the diagnostic method comprises at least one of a blood test, an imaging protocol, a biopsy, or a histopathological analysis.

In another particular embodiment of the above method, the method further comprises
(a) determining, by computer module, if a genome of the subject comprises the particular validated genome variant associated with the cancer,
wherein identifying that the genome of the subject comprises the particular validated genome variant associated with the cancer selects the subject as in need of at least one therapeutic regimen, wherein the therapeutic regimen comprises a protocol for reducing cancer cell number in the subject, wherein the protocol comprises at least one of:
   (i) a therapeutic agent used to treat the cancer;
   (ii) chemotherapy used to treat the cancer;
   (iii) radiation used to treat the cancer; or
   (iv) surgical resection of the cancer; and
(b) implementing the therapeutic regimen on the subject identified as having the genome comprising the particular validated genome variant associated with the cancer.

In another particular embodiment of the above method, the method further comprises
(a) obtaining a subject, wherein the subject has a preliminary diagnosis of the cancer, wherein the preliminary diagnosis is based on results from at least one diagnostic method for detecting the cancer in the subject; and
(b) determining, by computer module, if a genome of the subject comprises a particular validated genome variant associated with a cancer,
   wherein if the particular validated genome variant associated with the cancer is not detected in the subject's genome, the treatment regimen proposed for the cancer in the subject based on the preliminary diagnosis is not recommended, thereby reducing the frequency of ineffective treatment regimens of the cancer in the subject.

In another particular embodiment of the above method, the method further comprises
(a) obtaining a subject, wherein the subject has a preliminary diagnosis of the cancer, wherein the preliminary diagnosis is based on results from at least one diagnostic method for detecting the cancer in the subject; and
(b) determining, by computer module, if a genome of the subject comprises a particular validated genome variant associated with a cancer,
   wherein if the particular validated genome variant associated with the cancer is not detected in the subject's genome, the preliminary diagnosis of the cancer in the subject is identified as a false positive diagnosis of the cancer in the subject, thereby reducing the frequency of false positive diagnoses of the cancer in the subject.

In another aspect, a method is presented, comprising:
receiving, by a computing module, DNA sequencing data;
wherein the DNA sequencing data is representative of sequences from non-paired reads or paired reads with unsequenced DNA between them of a genome of a subject;
receiving, by the computing module, reference DNA analysis data for at least one DNA reference sequence;
wherein the reference DNA analysis data
   1) identifies each potential reference genome variant and
   2) comprises at least one of:
      i) a split-mapping identifying data,
      ii) an insertion/deletion (indel) identifying data,
      iii) a discordant mate identifying data, or
      iv) an unmapped mate identifying data;
evaluating, by computing module, each respective genome position of each respective sequence from the discordant paired reads of the genome to identify each respective validated genome variant, by simultaneously considering:
   i) the DNA sequencing data of the sequences from the discordant paired reads of the genome and
   ii) the reference DNA analysis data for the at least one DNA reference sequence;
wherein each respective validated genome variant is a genome variant confirmed based at least in part on the reference DNA analysis data;
wherein a speed to identify the same genome variant by simultaneously considering i) the DNA sequencing data and ii) the reference DNA analysis data is at least 1.5 fold faster than that of a speed to identify the same genome variant by analyzing the DNA sequencing data alone without the reference DNA analysis data.

In another particular embodiment of the above method, the particular genome variant is associated with a cancer. More particularly, the particular genome variant associated with a cancer is listed in Table 3.

In a particular embodiment of the above method, a particular validated genome variant is associated with a particular disease or a particular disorder. In a more particular embodiment of the method, the particular disease or the particular disorder is a cancer or a similar condition, wherein a diseased part of a body has a genotype different by one or more breakpoints from a healthy part of the body.

In another aspect, a method for selecting a subject in need of at least one of a monitoring method or diagnostic method and implementing the at least one monitoring method or diagnostic method is presented, wherein the monitoring or the diagnostic method relates to monitoring or diagnosing a cancer in the subject, the method comprising:
(a) obtaining a particular validated genome variant of a subject, by:
receiving, by a computing module, DNA sequencing data;
wherein the DNA sequencing data is representative of sequences from discordant paired reads of a genome of the subject;
receiving, by the computing module, reference DNA analysis data for at least one DNA reference sequence;
wherein the reference DNA analysis data
   1) identifies each potential reference genome variant and
   2) comprises at least one of:
      i) a split-mapping identifying data,
      ii) an insertion/deletion (indel) identifying data,
      iii) a discordant mate identifying data, or
      iv) an unmapped mate identifying data;
evaluating, by computing module, each respective genome position of each respective sequence from the discordant paired reads of the genome to identify the particular validated genome variant, by simultaneously considering:
   i) the DNA sequencing data of the sequences from the discordant paired reads of the genome and
   ii) the reference DNA analysis data for the at least one DNA reference sequence;
wherein the particular validated genome variant is a genome variant confirmed based at least in part on the reference DNA analysis data;
   wherein a speed to identify the same genome variant by simultaneously considering i) the DNA sequencing data and ii) the reference DNA analysis data is at least 1.5 fold faster than that of a speed to identify the same genome variant by analyzing the DNA sequencing data alone without the reference DNA analysis data;
wherein the cancer is listed in Table 3; and
(b) performing at least one of the monitoring method or the diagnostic method relating to monitoring or diagnosing the cancer in the subject, wherein if the performing the at least one monitoring method or the diagnostic method confirms a presence of the cancer in the subject, the subject is selected for a regimen comprising at least one additional monitoring method or diagnostic method.

In another aspect, a method for selecting a subject in need of a therapeutic regimen and treating the subject with the therapeutic regimen is presented, the method comprising:
(a) obtaining a particular validated genome variant of a subject, by:
receiving, by a computing module, DNA sequencing data;
wherein the DNA sequencing data is representative of sequences from discordant paired reads of a genome of the subject;
receiving, by the computing module, reference DNA analysis data for at least one DNA reference sequence;
wherein the reference DNA analysis data
   1) identifies each potential reference genome variant and
   2) comprises at least one of:
      i) a split-mapping identifying data,
      ii) an insertion/deletion (indel) identifying data,
      iii) a discordant mate identifying data, or
      iv) an unmapped mate identifying data;
evaluating, by computing module, each respective genome position of each respective sequence from the discordant paired reads of the genome to identify the particular validated genome variant, by simultaneously considering:
   i) the DNA sequencing data of the sequences from the discordant paired reads of the genome and
   ii) the reference DNA analysis data for the at least one DNA reference sequence;
wherein the particular validated genome variant is a genome variant confirmed based at least in part on the reference DNA analysis data;
   wherein a speed to identify the same genome variant by simultaneously considering i) the DNA sequencing data and ii) the reference DNA analysis data is at least 1.5 fold faster than that of a speed to identify the same genome variant by analyzing the DNA sequencing data alone without the reference DNA analysis data;
wherein the cancer and the therapeutic regimen for treating the cancer are listed in Table 3; and
(b) exposing the subject in need thereof to the therapeutic regimen, wherein the therapeutic regimen comprises a protocol for reducing cancer cell number in the subject, wherein the protocol comprises at least one of
   (i) a therapeutic agent used to treat the cancer;
   (ii) chemotherapy used to treat the cancer;
   (iii) radiation used to treat the cancer; or
   (iv) surgical resection of the cancer
      thereby selecting the subject in need of the therapeutic regimen and treating the subject with the therapeutic regimen.

In another aspect, a method for reducing ineffective treatment regimens of a cancer in a subject is presented, the method comprising:
(a) obtaining a subject having a preliminary diagnosis of the cancer, wherein the preliminary diagnosis is based on results from at least one diagnostic method for detecting the cancer in the subject; and
(b) obtaining a particular validated genome variant of the subject, by:
receiving, by a computing module, DNA sequencing data;
wherein the DNA sequencing data is representative of sequences from discordant paired reads of a genome of the subject;
receiving, by the computing module, reference DNA analysis data for at least one DNA reference sequence;
wherein the reference DNA analysis data
   1) identifies each potential reference genome variant and
   2) comprises at least one of:
      i) a split-mapping identifying data,
      ii) an insertion/deletion (indel) identifying data,
      iii) a discordant mate identifying data, or
      iv) an unmapped mate identifying data;
evaluating, by computing module, each respective genome position of each respective sequence from the discordant paired reads of the genome to identify the particular validated genome variant, by simultaneously considering:
   i) the DNA sequencing data of the sequences from the discordant paired reads of the genome and
   ii) the reference DNA analysis data for the at least one DNA reference sequence;
wherein the particular validated genome variant is a genome variant confirmed based at least in part on the reference DNA analysis data;
   wherein a speed to identify the same genome variant by simultaneously considering i) the DNA sequencing data and ii) the reference DNA analysis data is at least 1.5 fold faster than that of a speed to identify the same genome variant by analyzing the DNA sequencing data alone without the reference DNA analysis data;
wherein the cancer is listed in Table 3; and
wherein if the particular validated genome variant associated with the cancer is detected in the subject's genome, the preliminary diagnosis of the cancer in the subject is identified as a true positive diagnosis, and
wherein if the particular validated genome variant associated with the cancer is not detected in the subject's genome, the treatment regimen proposed to treat the cancer in the subject based on the presence of the variant is not recommended, thereby reducing the frequency of ineffective treatment regimens of the cancer in the subject.

In another aspect, a method for reducing a frequency of a false positive diagnoses of a cancer in a subject is presented, the method comprising:
(a) obtaining a subject having a preliminary diagnosis of the cancer, wherein the preliminary diagnosis is based on results from at least one diagnostic method for detecting the cancer in the subject; and
(b) obtaining a particular validated genome variant of the subject, by:
receiving, by a computing module, DNA sequencing data;
wherein the DNA sequencing data is representative of sequences from discordant paired reads of a genome of the subject;
receiving, by the computing module, reference DNA analysis data for at least one DNA reference sequence;
wherein the reference DNA analysis data
   1) identifies each potential reference genome variant and
   2) comprises at least one of:
      i) a split-mapping identifying data,
      ii) an insertion/deletion (indel) identifying data,
      iii) a discordant mate identifying data, or
      iv) an unmapped mate identifying data;
evaluating, by computing module, each respective genome position of each respective sequence from the discordant paired reads of the genome to identify the particular validated genome variant, by simultaneously considering:
   i) the DNA sequencing data of the sequences from the discordant paired reads of the genome and
   ii) the reference DNA analysis data for the at least one DNA reference sequence;
wherein the particular validated genome variant is a genome variant confirmed based at least in part on the reference DNA analysis data;
   wherein a speed to identify the same genome variant by simultaneously considering i) the DNA sequencing data and ii) the reference DNA analysis data is at least 1.5 fold faster than that of a speed to identify the same genome variant by analyzing the DNA sequencing data alone without the reference DNA analysis data;
wherein the cancer is listed in Table 3; and
wherein if the particular validated genome variant associated with the cancer is detected in the subject's genome, the preliminary diagnosis of the cancer in the subject is identified as a true positive diagnosis, and
wherein if the particular validated genome variant associated with the cancer is not detected in the subject's genome, the preliminary diagnosis of the cancer in the subject is identified as a false positive diagnosis of the cancer in the subject, thereby reducing the frequency of false positive diagnoses of the cancer in the subject.

As used herein, a “variant” can be any change in an individual nucleotide sequence compared to a reference sequence. The reference sequence can be a single sequence, a cohort of reference sequences, or a consensus sequence derived from a cohort of reference sequences.

In some embodiments, unlike currently available variant detection tools, embodiments of the present disclosure apply a unified decision making model considering multiple evidence types simultaneously to determine a likelihood of a variant at each genome position. Information utilized by the unified decision making model is collected at each reference base. Each read with a split mapping, indel, discordant mate, or unmapped mate contributes breakpoint evidence to each potential reference base breakpoint. Discordant pairs are identified based on abnormal read orientation or abnormal insert size. Insert size pertains to the original DNA fragment that was sequenced. If the mapping of a read pair suggests an insert size larger or smaller than expected, it is classified as an abnormal insert size. Determination of abnormal insert size is based on a sample of 10 million paired reads. Since insert size distributions tend to have right skewness, a rank-based method is used to determine abnormal insert size thresholds corresponding to 3 standard deviations from the median under a normal distribution (after outliers more than 5× the median insert size have been filtered). For simple cases such as a 2-base deletion within a read, there is one potential reference base start breakpoint and one potential reference base end breakpoint. Other cases may have less precise breakpoints, such as a read from a discordant deletion pair (abnormally large insert size). In this case, the exact breakpoint is unknown and a potential breakpoint is recorded for each reference base consistent with forming a concordant pair in the sample, where a concordant pair corresponds to insert sizes≥iₘᵢₙ and ≤iₘₐₓ, where iₘᵢₙ and iₘₐₓ represent the minimum and maximum insert size thresholds, respectively (FIG. 3).

Using the deletion example in FIG. 3, a breakpoint distant from both reads would necessitate an insert size that is too large to be consistent with a concordant pair (and the source DNA fragment), and thus would not be a potential breakpoint. When soft-clipping (≥5 bases) or a split-read (each mapped split≥20 bases) occurs in the potential breakpoint region, the reference base immediately adjacent to the soft-clipping or split-read is recorded as a potential breakpoint and other potential breakpoints are recorded with half-weighting. This enables base resolution of breakpoints while limiting a single aberrant read mapping from misidentifying the true breakpoint.

For each reference base, breakpoint evidence is stored for each indel and SV type (deletion, duplication, etc.). For each potential breakpoint of a read supporting an indel or SV, the corresponding indel or SV length is compared with lengths of existing clusters that have the same indel or SV type. Breakpoint evidence for a cluster is incremented if the lengths are close, i.e.,

$$\vert {L}_{bc} - {L}_{disc} \vert \leq ( {i}_{ma x} - {i}_{min} + {i}_{median} - 2 {L}_{r} ) ( 1 + \frac{1}{{x}_{bc}} )$$

where L_{bc} is the mean indel or SV length for the breakpoint cluster, L_{disc} is the length of the indel or SV pertaining to the candidate read, Lᵣ is the read length, x_{bc} is the number of previously recorded reads supporting the breakpoint cluster, and iₘₐₓ and iₘᵢₙ are the maximum and minimum concordant pair lengths, respectively. If a candidate read does not fit in any existing breakpoint clusters, a new cluster is created.

For each reference base, a mismapping probability, p_{bc}, is calculated for each possible SNV, indel, and SV. p_{bc} is the binomial probability of at least X_{bc} reads supporting the breakpoint cluster given n_{bc} read depth and a mapping quality threshold m. Thus, p_{bc} indicates the likelihood that all of the supporting reads are mismappings. Read depth includes all mapped reads, unsequenced segments between concordant pairs, and potential breakpoints, and thus is an estimate of physical coverage. Physical coverage provides a more comprehensive representation of genome coverage than read coverage. It also helps define deletion and duplication breakpoints when soft-clipping is unavailable as a decrease in coverage will affect breakpoint probability estimates. The mapping quality threshold m indicates the probability of a read mismapping:

$$p = {10}^{- \frac{m}{10}}$$

Thus, p_{bc} is given as:

$${p}_{bc} = Pr ( X \geq x ) = 1 - {\sum }_{k = 0}^{x - 1} ( \begin{matrix}n \\ k\end{matrix} ) {p}^{k} {q}^{n - k}$$

where q=1−p. To reduce computational time, binomial probability tables are precomputed and stored as data files.

In some embodiments, the present disclosure utilizes the findings of one type of variant to further inform the computational protocol about possible effects on finding other types of variants. In some embodiments for example, the present disclosure factors detected changes in single-nucleotide variant (SNV) allele frequency as indicators of a possible structural variant (SV). In some embodiments for example, a detected structural variant (SV) affects in its vicinity, the parameters used for single-nucleotide variant (SNV) detection.

In some embodiments, the present disclosure describes a computational protocol for identifying somatic rearrangements in cancer genomes. In some embodiments, the computational protocol includes: (a) finding discordant paired reads (e.g. with abnormal orientation or abnormal insert size); (b) simultaneously incorporating additional mapping information (e.g. soft-clipping) and elements from split-read and read-depth methods (e.g. sequence bias normalization) with the discordant paired reads; and (c) evaluating the discordant paired reads with the mapping information and with elements from split-read and read-depth methods to predict a breakpoint present in the cancer genome but absent in the normal genome.

In some embodiments, the computational protocol begins by (a) finding discordant paired reads (e.g. with abnormal orientation or abnormal insert size). In some embodiments, the mapping algorithm, such as Burrows-Wheeler Aligner (BWA), reports the orientation for each mapped read. The orientation for each mapped read may be forward (i.e. mapped to the forward strand of the reference genome) or reverse (i.e. mapped to the reverse strand of the reference genome). For a normal orientation, the read mapped to the left-most reference location has forward orientation and the read mapped to the right-most reference location has reverse orientation. This orientation is referred to as forward-reverse. Any other orientation is considered abnormal (e.g., forward-forward, reverse-reverse, reverse-forward). An abnormal insert size indicates a pair of reads that has mapped to a reference genome and has a mapped distance (e.g. estimated insert size) that is significantly larger or smaller than normal. Determination of abnormal insert size is based on a sample of 10 million paired reads. Since insert size distributions tend to have right skewness, a rank-based method is used to determine abnormal insert size thresholds corresponding to 3 standard deviations from the median under a normal distribution (after outliers more than 5× the median insert size have been filtered).

Next, the computational protocol simultaneously incorporates additional mapping information (e.g. soft-clipping) and elements from split-read and read-depth methods (e.g. sequence bias normalization) with the discordant paired reads. Each read (split-read or mate-unmapped read, soft-clipped read) or read pair (discordant pair) contributes one unit of evidence (½ unit for soft-clipped and split-read read breakpoints more than one base from the read's mapped bases). All other reads and unsequenced regions that may constitute physical coverage (coverage of the original DNA fragments) contribute one unit of non-supporting evidence.

Next, the computational protocol evaluates the discordant paired reads with the mapping information and with elements from split-read and read-depth methods to predict a breakpoint present in the cancer genome but absent in the normal genome.

In some embodiments, the present disclosure describes a method for identifying somatic rearrangements in cancer genomes, the method comprising: using a programmed computer processor or specially-designed hardware to: (a) find discordant paired reads (e.g. with abnormal orientation or abnormal insert size); (b) simultaneously incorporate additional mapping information (e.g. soft-clipping) and elements from split-read and read-depth methods (e.g. sequence bias normalization) with the discordant paired reads; and (c) evaluate the discordant paired reads with the mapping information and with elements from split-read and read-depth methods to predict a breakpoint present in the cancer genome but absent in the normal genome. In some embodiments, the programmed computer processor is a binary or pre-compiled executable algorithm designed for linux systems. Any description herein of specific hardware is not intended to be limiting to the type of hardware that is suitable to run the algorithm described herein. In some embodiments, analysis of large genomes, such as the human genome, may require approximately 128-1024 GB of RAM memory.

As used herein, the term discordant paired-end reads refers to reads mapped to the reference sequence in a way indicative of a structural variation. These discordant reads are clustered to provide high confidence for the occurrence of each structural variation. As used herein, soft clipping refers to unmatched fragment in a partially mapped read. As used herein, sequencing depth (also known as read depth) describes the number of times that a given nucleotide in the genome has been contained in sequenced reads and unsequenced DNA between paired reads in an experiment.

In some embodiments, the computational protocol further comprises calculating a probabilistic score based on paired-read and read-depth information to evaluate breakpoint potential at each base. As described above,

In some embodiments, the computational protocol will find variants by analyzing multiple samples (e.g., patient cohorts) simultaneously and using combined evidence to further improve accuracy. In some embodiments, to find variants by analyzing multiple samples simultaneously and using combined evidence, the computational protocol will extend GROM's score at each genome position, produced for all variants (SNVs, indels, SVs, CNVs), to take into account co-occurrence of breakpoints in several samples and thus provide a robust “weak evidence” metric. In some embodiments, a single sample score will be S=Σ_{T}w(L_{T}), where T is a type of next-generation sequencing (NGS) evidence (e.g. split read, discordant read pair, or the like) in a given genome location and w(L_{T}) is a weighting function. For m samples with available NGS evidence for a breakpoint out of n total samples, the score will become S=E(m,n)Σ_{T}w(L_{T}), where E(m,n) is a function that favors co-occurring breakpoints across samples. For example, E(m,n) can be scaled as m!n/(n−m+1) or as an exponent. Information from previous studies will be able to quickly improve variant detection sensitivity in the new study without the need for extensive re-analysis. In some embodiments, the computational protocol will use “weak evidence” (e.g. evidence that is insufficient to call a variant when only one genome is analyzed) for variants with similar genome positions in multiple samples. Weak evidence occurs when there are one or more discordant pairs, soft-clipped reads, or split-reads supporting a variant but the evidence is not enough (i.e. does not reach thresholds for criteria such as the mismapping probability, p_{bc}) to predict a variant.

In some embodiments, the computational protocol will find associations of SVs (in non-coding regions) with changes in expression of nearby genes. In some embodiments, finding associations of SVs will be done on multiple samples using additional RNA-Seq data for the same samples. In some embodiments, the exemplary method of this disclosure are configured to generate new disease biomarkers (represented by SVs, where the presence of a causal SV in a given region can then be used as a diagnostic marker using PCR and similar region-directed technologies for new patients, thereby avoiding costly sequencing) and targets (represented by SV-affected genes) for further translational validation.

In some embodiments, if the computational protocol finds a variant inactivating any gene in that network, any connected gene can be detected as a target for a given tumor genome. Some of these target genes have drugs inhibiting them. Taking these drugs can kill a tumor, resulting in personalized patient treatment. In some embodiments, the computational protocol of the present disclosure can be a decisive factor due to (i) superior accuracy needed to detect if a variant affects a gene and (ii) practical implementation of the “synthetic lethal” network.

### BRIEF DESCRIPTION OF THE DRAWINGS

Embodiments of the present invention, briefly summarized above and discussed in greater detail below, can be understood by reference to the exemplary embodiments of the invention depicted in the appended drawings. It is to be noted, however, that the appended drawings illustrate only exemplary embodiments of this invention and are therefore not to be considered limiting of its scope, for the invention may admit to other equally effective embodiments.

FIG. 1 depicts exemplary variants within a genome, in accordance with some embodiments of the present disclosure.

FIG. 2 depicts an exemplary workflow of a computational protocol in accordance with some embodiments of the present disclosure.

FIG. 3A-F depicts an outline of multi-sample variant visualization in accordance with some embodiments of the present disclosure.

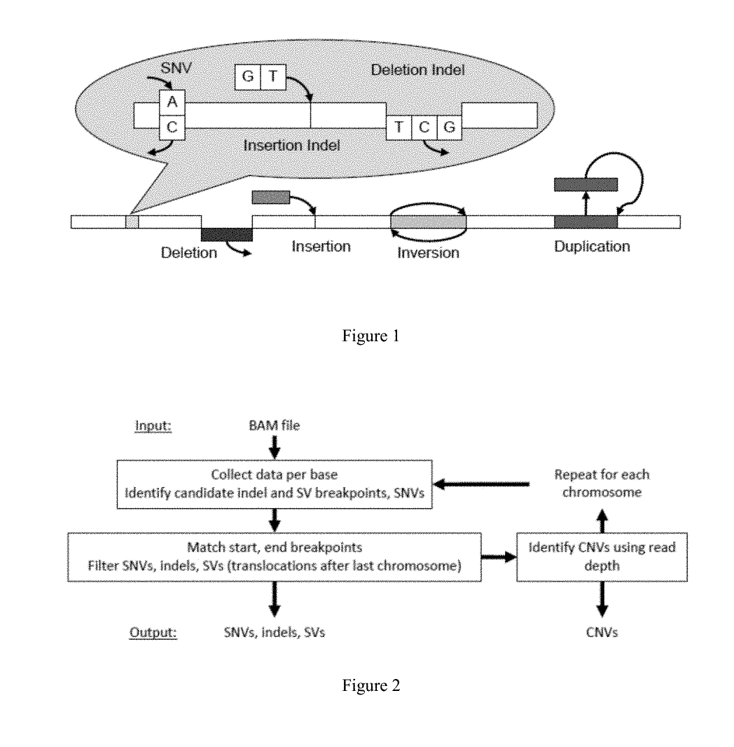

To facilitate understanding, identical reference numerals have been used, where possible, to designate identical elements that are common to the exemplary figures. The exemplary figures are not drawn to scale and may be simplified for clarity. It is contemplated that elements and features of one embodiment may be beneficially incorporated in other embodiments without further recitation.

### DETAILED DESCRIPTION

The present invention can be further explained with reference to the included drawings, wherein like structures are referred to by like numerals throughout the several views. The drawings shown are not necessarily to scale, with emphasis instead generally being placed upon illustrating the principles of the present invention. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention can become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the present invention is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though they may. Furthermore, the phrases “in another embodiment” and “in some other embodiments” as used herein do not necessarily refer to a different embodiment, although they may. Thus, as described below, various embodiments of the invention may be readily combined, without departing from the scope or spirit of the invention. Further, when a particular feature, structure, or characteristic is described in connection with an implementation, it is submitted that it is within the knowledge of one skilled in the art to effect such feature, structure, or characteristic in connection with other implementations whether or not explicitly described herein.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “runtime” corresponds to any behavior that is dynamically determined during an execution of a software application or at least a portion of software application.

In some embodiments, the programmed computing systems with associated devices are configured to operate in the distributed network environment, communicating over a suitable data communication network (e.g., the Internet, etc.) and utilizing at least one suitable data communication protocol (e.g., IPX/SPX, X.25, AX.25, AppleTalk™, TCP/IP (e.g., HTTP), etc.). Of note, the embodiments described herein may, of course, be implemented using any appropriate hardware and/or computing software languages. In this regard, those of ordinary skill in the art are well versed in the type of computer hardware that may be used, the type of computer programming techniques that may be used (e.g., object oriented programming), and the type of computer programming languages that may be used (e.g., C++, Objective-C, Swift, Java, Javascript). The aforementioned examples are, of course, illustrative and not restrictive.

The material disclosed herein may be implemented in software or firmware or a combination of them or as instructions stored on a machine-readable medium, which may be read and executed by one or more processors. As used herein, the machine-readable medium may include any medium and/or mechanism for storing or transmitting information in a form readable by a machine (e.g., a computing device). By way of example, and not limitation, the machine-readable medium may comprise computer readable storage media, for tangible or fixed storage of data, or communication media for transient interpretation of code-containing signals. Machine-readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Machine-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, flash memory storage, or any other physical or material medium which can be used to tangibly store the desired information or data or instructions, including but not limited to electrical, optical, acoustical or other forms of propagated signals (e.g., carrier waves, infrared signals, digital signals, etc.), and which can be accessed by a computer or processor.

In another form, a non-transitory article, such as non-volatile and non-removable computer readable media, may be used with any of the examples mentioned above or other examples except that it does not include a transitory signal per se. It does include those elements other than a signal per se that may hold data temporarily in a “transitory” fashion such as RAM and so forth. In some embodiments, the present invention may rely on one or more distributed and/or centralized databases (e.g., data center).

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

As used herein, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

As depicted in FIG. 2, a computational protocol, in accordance with some embodiments of the present disclosure, simultaneously collects data from a Binary Alignment Map (BAM) file for each reference base and identifies candidate breakpoints and SNVs in one passthrough of a BAM file. A BAM file is the output from a mapping algorithm (BWA), usually a text-format (SAM) that has subsequently been converted to a binary format (BAM). A BAM format is the industry standard, and is the only format accepted by the present invention. Next, after each chromosome data is collected, SNVs are filtered; the start and end of breakpoints are matched and filtered for each indel and SV (excluding translocations). Next, copy number variants (CNVs) are identified.

CNVs are identified by regions of the genome with abnormal read coverage, with low coverage indicating a deletion and high coverage indicating an amplification. In some embodiments, the output is a union set from two pipelines that differ based on the inclusion or exclusion of a pre-filtering step, excessive coverage masking.

Exemplary methods for identifying/determining nucleotide content include the following:

Excessive Coverage Masking:

Complex and repetitive segments are common in the human genome and can complicate CNV detection. Such high read coverage may result in false positives and also reduce CNV sensitivity in less complex regions. In some embodiments, a two-pipeline approach is used to detect CNVs in complex and repetitive segments and improve sensitivity in less complicated regions. In the first pipeline, clusters of blocks (10,000 base segments) with high read coverage (default: >2× chromosome average) are masked prior to CNV detection. A cluster is defined as a section of the genome where >25% of the blocks have high read coverage and a minimum of four blocks have high read coverage. In the second pipeline, CNVs are detected on the unmasked genome to identify CNVs in complex regions. A union set of predicted CNVs is output following the two pipelines. Many false positives may be produced from spikes in read coverage, particularly for the unmasked genome. Thus during later steps in the pipeline, read coverage greater than twice the chromosome average is adjusted (described in GC Bias Normalization below at paragraph [0077]).

GC Weighting:

Variation in the GC content of genome regions affects read coverage produced by NGS platforms. A post-sequencing approach used by many RD algorithms, such as CNVnator and RDXplorer, is to bin genome regions by GC content and adjust the average read depth of each bin to the average read depth of the genome, referred to as GC bias normalization.

The first step of this approach is to calculate GC content of genome regions. RD algorithms often divide a chromosome into regions, referred to as windows, of a fixed size and estimate read depth in each window by counting reads within the window. GC content for a window is calculated from the proportion of reference sequence G and C bases within the window. Previous studies have identified PCR bias as the main contributor to GC bias in NGS. Thus, reference bases outside a window may affect read coverage within a window, especially for long reads and paired-end reads. Previous studies have shown a higher correlation between GC content and read depth when considering the GC content of the entire PCR-replicated DNA fragment rather than the sequenced segment. In some embodiments, based on these observations, a GC weighting method considers all bases within an average insert size. In some embodiments, to maximize sensitivity, GC weighting is not calculated for a window of bases; instead GC weighting is calculated for each base i as hᵢ=Σwⱼaⱼ/Σwⱼ, where j is a base that may affect read depth for base i, wⱼ is the weight of base j and is equivalent to the sum of average inserts with unique starting locations and that overlap base j and base i, and aⱼ is 1 if base j is a G or C and 0 otherwise. For single-end reads, the insert size is equivalent to read length.

GC Bias Normalization:

For single-end reads, the insert size is equivalent produced by NGS platforms as a result of variation in the GC content of genome regions. Many RD algorithms, such as CNVnator and RDXplorer, bin genome regions (windows) by GC content and adjust the average read depth of each bin to the average read depth of the genome:

r_{i,norm}=rᵢm/m_{GC} (1)

where r_{i,norm} is the read coverage of a window after normalization, rᵢ is the read coverage of window i prior to normalization, m is the global mean read coverage of all windows in the genome, and m_{GC} is the mean read coverage of all windows with similar GC content. However, utilizing this method, differences in variance may remain after GC bias correction. From this observation, it can be expected that methods using this approach will over-predict CNVs when a GC region has high variance and under-predict CNVs when a GC region has low variance. A quantile normalization approach is used to correct for variance across bins of GC weighted bases. For this approach, bases are ranked in each bin based on read depth and a rank proportion pᵢ is calculated for each base i using:

pᵢ=Rᵢ/n if 2 Rᵢ≤n

pᵢ=(n−Rᵢ)/n if 2 Rᵢ>n (2)

where Rᵢ is the read depth rank for base i and n is a count of bases with a particular GC weighting. When Rᵢ is 0 (for 2 Rᵢ≤in) or n−Rᵢ is 0 (for 2 Rᵢ>n), the numerator in Eq. (2) is set to 0.5. Subsequently, pᵢ is converted to standard deviation units, xᵢ, using a pre-computed normal distribution table. Note when n is identical for all GC bins and there are no read depth ties within a GC bin, each bin distribution will have identical statistical properties, including mean and variance, after quantile normalization. Statistical properties of quantile normalized distributions may vary across GC bins when n varies, however this effect is negligible when n is large. In some embodiments a GC bin has at least 100 bases. A normalized read depth as in Eq. (1) is not produced because it is not necessary for further analysis. Instead, read depth in standard deviation units is used. To reduce false positives, read coverage greater than twice the chromosome average is adjusted by averaging the rank of the observed read coverage and the rank of read coverage equivalent to twice the chromosome average read coverage. CNVs may occur in low mapping quality regions; however, read coverage distributions tend to differ between low mapping quality and high mapping quality regions. To compensate for variation of read coverage distributions with mapping quality, the average mapping quality for each window is calculated separate distributions for low mapping quality (default: <5) and high mapping quality windows are created. The nature of the read depth distribution for NGS data has not been clearly defined. A rank-based approach does not assume a specific distribution and is less affected by outliers when compared to parametric methods.

Dinucleotide Repeat Bias Normalization

Repeat bias has been observed with NGS technologies. Additionally, these biases may vary with sequencing technology and genomes. In some embodiments, dinucleotide repeat biases are detected and a quantile normalization method is utilized in the respective genomic regions. Dinucleotide repeats with average read coverage that is more than 1.5 standard deviations below the genome average read coverage, and vice versa (genome coverage more than 1.5 standard deviations above dinucleotide coverage), are considered biased. For a biased dinucleotide repeat, we use a quantile normalization approach similar to our GC bias normalization, except Rᵢ is the read depth rank of occurrence i of a particular dinucleotide repeat.

From this read depth in standard deviation units for each biased dinucleotide repeat occurrence is obtained. For regions further from a repeat, the separate sample distributions in 10 base increments to adjust for the decreasing influence of repeat bias are created. Thus, bases are bined by distance from the repeat, in contrast to binning by GC weighting as described above in paragraph [0031]. Repeat bias normalization is applied within a distance of half-insert size from biased dinucleotide repeats. For genomic regions with dinucleotide repeat bias, dinucleotide repeat bias normalization replaces GC bias normalization.

Sliding Window CNV Search:

RD methods typically suffer from reduced breakpoint resolution compared to other methods, such as split-read. One reason for low resolution is fixed-size, non-overlapping windows. In some embodiments, sliding windows that sequentially increase in one-base increments are employed to improve breakpoint resolution. Fixed-size, non-overlapping windows also reduce sensitivity when CNVs start or end near the center of a non-overlapping window. Using sliding windows, improve sensitivity to CNVs regardless of start or end points. Additionally, creating distributions for incremental window sizes improves sensitivity on a range of CNV sizes. In some embodiments, GC bias or, if necessary, dinucleotide repeat bias for each base is normalized. In some embodiments, normalized bases are combined into windows by averaging standard deviation units of all bases in a window. Since the means and variances of the bases have been normalized with respect to GC bias or dinucleotide repeat bias, GC and dinucleotide bias are not associated with the windows. For each window size, a set of windows is sampled from the dataset and a read depth mean and standard deviation is obtained. Then, base positions are identified with abnormal read coverage≥1.3r_{ave,h} for duplications or ≤0.70r_{ave,h} for deletions (for diploids) as potential breakpoints, where r_{ave,h} is the average read depth for bases with h weighted GC content. If at least half of the bases have abnormal coverage for a minimum window size, w_{l,min} (default=100) beginning at a potential breakpoint j, a z-score, z, is calculated based on a sample distribution of read depths for w_{l,min} and the read depth of a window i having size w_{l,min} and beginning at j. Several parameters affect calling CNVs as outlined below (and they can potentially be modified by a user). A CNV is called if z<α, (default: α=1×10−6). In some embodiments, the window size is increased in one-base increments and z is recalculated to either extend or detect a CNV until a maximum window size w_{l,max} (default=10,000) is reached. If no CNV has been detected, the statistical testing is repeated at the next potential breakpoint. Attempts to extend or detect a CNV will end before reaching w_{l,max} if less than half the bases have abnormal read coverage (≥1.3 or ≤0.70r_{ave,h} for diploids). If a CNV was found and w_{l,max} has been reached, the CNV may be extended by sliding a window of size w_{l,max} and recalculating z. Attempts to extend a CNV continue until thresholds related to read coverage and distance from the CNV end breakpoint have been reached.

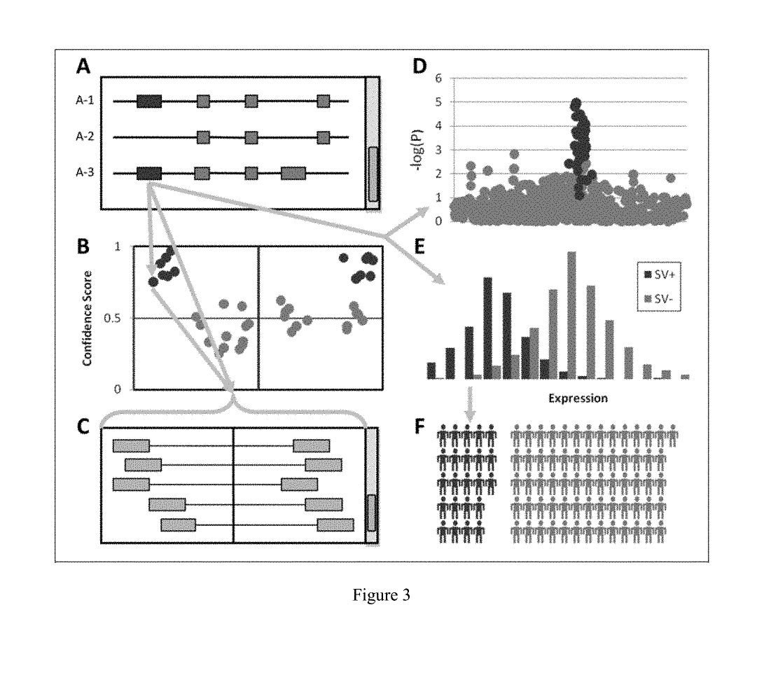

FIG. 3 depicts an outline of multi-sample variant visualization in accordance with some embodiments of the present disclosure. FIG. 3A provides an overview of variants across patient genomes (e.g. A-1 to A-3). By selecting a variant (e.g. A-3), a user can open windows for further analysis, including multi-sample breakpoint plot (FIG. 3B), single-sample reads viewer (FIG. 3C,), gene expression association results along the genome (FIG. 3D) and gene expression level distributions for patient subgroups (FIG. 3E). The darker shade color across all panels corresponds to a hypothetical disease-relevant SV and patient genomes containing it (FIG. 3F). The start and end breakpoints of variants in FIG. 3B are shown as connected clickable dots colored by a feature, such as patient subgroups. The X-axis shows reference position, and the y-axis gives a confidence score. The single-sample read viewer of FIG. 3C enables a user to view reads at each breakpoint in adjacent windows. Manhattan plot peaks of expression association links SVs with the corresponding genes (See FIG. 3D), the expression of which is expected to be different between patient subgroups (See FIGS. 3E and 3F).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Software may refer to 1) libraries; and/or 2) software that runs over the internet or whose execution occurs within any type of network. Examples of software may include, but are not limited to, software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor.

Four commonly used algorithms for detection of genome variants are: GATK-HC, SAMtools, LUMPY, and Manta. A comparison of the four commonly used algorithms versus embodiments of the present disclosure using two extensively validated human whole genome sequencing (WGS) datasets, NA12878 “platinum” genome and HX1, a Chinese genome, resulted in embodiments of the present disclosure exhibiting the highest SNV and insertion indel sensitivity and precision and the highest deletion indel sensitivity when compared to GATK-HC and SAMtools, and superior deletion and duplication detection compared to LUMPY and Manta. Additionally, embodiments of the present disclosure exhibited the highest sensitivity and precision in all inversion and insertion metrics. Further, embodiments of the present disclosure analyzed a 50× WGS human dataset (NA12878) on commonly available computer hardware in 11 minutes, more than an order of magnitude faster than a combination of tools together detecting the same types of variants. Embodiments of the present disclosure proved to be 1.7× (NA12878) and 2.1× (HX1) faster than the next fastest algorithm, Manta.

Moreover, embodiments of the present disclosure reveal that DNA sequencing systems and methods described herein allow for paired and non-paired reads. Accordingly, embodiments of the present disclosure are compatible with various sequencing platforms, including Illumina sequencing platforms (mostly designed for paired reads) and other sequencing platforms such as, e.g., Pacbio, Oxford Nanopore (mostly designed for non-paired long reads). The universal compatibility of embodiments described herein underscores yet another distinct and improved feature of the DNA sequencing systems and methods described herein.

Table 1 below depicts a comparison of embodiments of the present disclosure and the above four commonly used algorithms' variant detection accuracy and run time. Performance based on sensitivity and precision rankings (1=highest, 3=lowest) in seven variant types averaged across a total of 18 benchmarks on validated variants (15 benchmarks for SVs).

TABLE 1

| Algorithm | | GATK-HC | SAMtools | LUMPY | Manta | GROM |
|---|---|---|---|---|---|---|
| SNV | | 2 | 3 | — | — | 1 |
| Indel | Deletion | 1 | 3 | — | — | 1 |
| | Insertion | 2 | 3 | — | — | 1 |
| | Deletion | — | — | 2 | 3 | 1 |
| SV | Duplication | — | — | 2 | 2 | 1 |
| | Insertion | — | — | — | 2 | 1 |
| | Inversion | — | — | 3 | 2 | 1 |

| | | | | | |
|---|---|---|---|---|---|
| Run Time | 4 | 5 | 3 | 2 | 1 |

Table 2 depicts run time comparison with and without duplicate filtering. Sambamba was chosen for duplicate filtering due to its multithreading capability. Embodiments of the present disclosure used a built-in duplicate filter. For other tools duplicate filtering was performed using SAMtools for single-threaded tests and Sambamba for multi-threaded tests.

TABLE 2

| | NA12878 | HX1 |
|---|---|---|
| | Single-threaded Run Time | |
| | (with/without duplicate filtering, minutes)^{a} | |
| Algorithm | | |
| GATK-HC | 3021/2413 | 6139/5222 |
| SAMtools | 3946/3338 | 5485/4569 |
| LUMPY | 1255/647 | 1570/653 |
| Manta | 993/385 | 1433/517 |
| GROM | 211/222 | 235/248 |
| | Multi-threaded Run Time | |
| | (with/without duplicate filtering, minutes)^{b} | |
| Algorithms | | |
| GATK-HC plus Manta | 794/684 | 1072/944 |
| GROM | 11/12 | 38/40 |

TABLE 3

| | | | |
|---|---|---|---|
| Gene | Alterations | Disease | Drugs |

Level 1
FDA-approved: 17 genes, 39 Alterations

| | | | |
|---|---|---|---|
| ABL1 | BCR-ABL1 Fusion | B -Lymphoblastic | Imatinib, Dasatinib |
| | | Leukemia/Lymphoma | |
| ABL1 | BCR-ABL1 Fusion | Chronic Myelogenous Leukemia | Imatinib, Nilotinib, Dasatinib |
| ALK | Fusions | Non-Small Cell Lung Cancer | Crizotinib, Ceritinib, Alectinib, |
| | | | Brigatinib |
| BRAF | V600 | Erdheim-Chester Disease | Vernurafenib |
| BRAF | V600E | Anaplastic Thyroid Cancer | Dabrafenib + Trametinib |
| BRAF | V600E | Melanoma | Dabrafenib, Vemurafenib |
| BRAF | V600E | Non-Small Cell Lung Cancer | Dabrafenib + Trametinib |
| BRAF | V600E, V600K | Melanoma | Dabrafenib + Trametinib, |
| | | | Trametinib, |
| | | | Cobimetnib + Vernurafenib, |
| | | | Binimetinib + Encorafenib |
| BRCA1 | Oncogenic Mutations | Ovarian Cancer | Rucaparib, Niraparib |
| BRCA2 | Oncogenic Mutations | Ovarian Cancer | Rucaparib, Niraparib |
| EGFR | Exon 19 deletion, | Non-Small Cell Lung Cancer | Erlotinib, Gefitinib, Afatinib, |
| | Exon 19 | | Osimertinib, Dacomitinib |
| | deletion/insertion, | | |
| | L858R | | |
| EGFR | Kinase Domain | Non-Small Cell Lung Cancer | Afatinib, Erlotinib, Gefitinib |
| | Duplication, M277E, | | |
| | A750P, G719, Exon | | |
| | 19 insertion, L747P, | | |
| | E709_T710delinsD, | | |
| | E709K, L833V, | | |
| | S768I, L861, | | |
| | A763_Y764insFQEA | | |
| EGFR | T790M | Non-Small Cell Lung Cancer | Osimertinib |
| ERBB2 | Amplification | Breast Cancer | Trastuzumab, Ado-Trastuzumab |
| | | | Emtansine, Lapatinib, |
| | | | Pertuzumab + Trastuzumab, |
| | | | Lapatinib + Trastuzurnab, |
| | | | Neratinib |
| ERBB2 | Amplification | Esophagogastric Cancer | Trastuzumab |
| IDH1 | Oncogenic Mutations | Acute Myeloid Leukemia | Ivosidenib |
| IDH2 | R140Q, R172 | Acute Myeloid Leukemia | Enasidenib |
| KIT | Exon 17 mutations | Gastrointestinal Stromal Tumor | Regorafenib |
| KIT | Oncogenic Mutations | Gastrointestinal Stromal Tumor | Imatinib, Sunitinib, Regorafenib |
| KIT | T670I, V654A | Gastrointestinal Stromal Tumor | Sunitinib, Regorafenib |
| KRAS | Wildtype | Colorectal Cancer | Cetuximab, Panitumumab, |
| | | | Regorafenib |
| Other | Microsatellite | All Solid Tumors | Pembrolizumab |
| Biomarkers | Instability-High | | |
| Other | Microsatellite | Colorectal Cancer | Nivolumab |
| Biomarkers | Instability-High | | |
| PDGFRA | FIP1L1-PDGFRA | Chronic Eosinophilic Leukemia, | Imatinib |
| | Fusion | NOS | |
| PDGFRA | Fusions | Myelodysplastic/Myeloproliferative | Imatinib |
| | | Neoplasms | |
| PDGFRB | Fusions | Dermatofibrosarcoma Protuberans | Imatinib |
| PDGFRB | Fusions | Myelodysplastic/Myeloproliferative | Imatinib |
| | | Neoplasms | |
| ROS1 | Fusions | Non-Small Cell Lung Cancer | Crizotinib |
| TSC1 | Oncogenic Mutations | CNS Cancer | Everolimus |
| TSC2 | Oncogenic Mutations | CNS Cancer | Everolimus |

Level 2
Standard care: 10 Genes, 22 Alterations

| | | | |
|---|---|---|---|
| ALK | Fusions | Inflammatory Myofibroblastic | Crizotinib, Ceritinib |
| | | Tumor | |
| BRCA1 | Oncogenic Mutations | Ovarian Cancer | Olaparib |
| BRCA2 | Oncogenic Mutations | Ovarian Cancer | Olaparib |
| CDK4 | Amplification | Dedifferentiated Liposarcoma | Abemaciclib, Palbociclib |
| CDK4 | Amplification | Well-Differentiated Liposarcoma | Abemaciclib, Palbociclib |
| KIT | Exon 17 mutations | Gastrointestinal Stromal Tumor | Sorafenib |
| KIT | Oncogenic Mutations | Melanoma | Imatinib |
| KIT | Oncogenic Mutations | Thymic Tumor | Sunitinib, Sorafenib |
| MET | Amplification | Renal Cell Carcinoma | Cabozantinib |
| MET | D1010H, D1010N, D1010Y, Exon | Non-Small Cell Lung Cancer | Crizotinib |
| | 14 splice mutation, Y1003C, | | |
| | Y1003F, Y1003N, Amplification | | |
| PDGFRA | D842V | Gastrointestinal Stromal Tumor | Dasatinib |
| PDGFRA | Oncogenic Mutations | Gastrointestinal Stromal Tumor | Imatinib |
| RET | Fusions | Non-Small Cell Lung Cancer | Cabozantinib, |
| | | | Vandetanib, LOXO-292 |
| TSC1 | Oncogenic Mutations | Renal Cell Carcinoma | Eyerolimus |
| TSC2 | Oncogenic Mutations | Renal Cell Carcinoma | Everolimus |

Level 3
Clinical Evidence: 26 Genes, 47 Alterations

| | | | |
|---|---|---|---|
| AKT1 | E17K | Breast Cancer | AZD5363 |
| AKT1 | E17K | Endometrial Cancer | AZD5363 |
| AKT1 | E17K | Ovarian Cancer | AZD5363 |
| ALK | G1202R | Non-Small Cell Lung | Lorlatinib |
| | | Cancer | |
| ARAF | Oncogenic Mutations | Histiocytosis | Sorafenib |
| ARAF | Oncogenic Mutations | Non-Small Cell Lung | Sorafenib |
| | | Cancer | |
| BRAF | Fusions | Melanoma | Cobimetinib, Trametinib |
| BRAF | Fusions | Ovarian Cancer | Cobimetinib, Trametinib |
| BRAF | K601, L597 | Melanoma | Trametinib |
| BRAF | V600 | Colorectal Cancer | Dabrafenib + Panitumumab + |
| | | | Trametinib |
| EGFR | Exon 20 insertion | Non-Small Cell Lung | Poziotinib |
| | | Cancer | |
| ERBB2 | Oncogenic Mutations | Breast Cancer | Neratinib |
| ERBB2 | Oncogenic Mutations | Non-Small Cell Lung | Neratinib |
| | | Cancer | |
| ERCC2 | Oncogenic Mutations | Bladder Cancer | Cisplatin |
| ESR1 | Oncogenic Mutations | Breast Cancer | AZD9496, Fulvestrant |
| FGFR1 | Amplification | Lung Squamous Cell | AZD4547, BGJ398, Debio1347, |
| | | Carcinoma | Erdafitinib |
| FGFR2 | Fusions | Bladder Cancer | AZD4547, BGJ398, Debio1347, |
| | | | Erdafitinib |
| FGFR2 | Fusions | Cholangiocarcinoma | AZD4547, BGJ398, Debio1347, |
| | | | Erdafitinib |
| FGFR3 | Fusions, G370C, | Bladder Cancer | AZD4547, BGJ398, Debio1347, |
| | G380R, K650, R248C, | | Erdafitinib |
| | 5249C, 5371C, | | |
| | Y373C | | |
| FLT3 | Internal tandem | Acute Myeloid Leukemia | Sorafenib |
| | duplication | | |
| HRAS | Oncogenic Mutations | Head and Neck Squamous | Tipifarnib |
| | | Cell Carcinoma | |
| JAK2 | PCM1-JAK2 Fusion | Chronic Eosinophilic | Ruxolitinib |
| | | Leukemia, NOS | |
| KIT | D816 | Mastocytosis | Avapritinib |
| MAP2 | Oncogenic Mutations | Histiocytosis | Cobimetinib, Trametinib |
| K1 | | | |
| MAP2 | Oncogenic Mutations | Low-Grade Serous Ovarian | Cobimetinib, Trametinib |
| K1 | | Cancer | |
| MAP2 | Oncogenic Mutations | Melanoma | Cobimetinib, Trametinib |
| K1 | | | |
| MAP2 | Oncogenic Mutations | Non-Small Cell Lung | Cobimetinib, Trametinib |
| K1 | | Cancer | |
| MDM2 | Amplification | Liposarcoma | RG7112, DS-3032b |
| MET | D1010H, D1010N, | Non-Small Cell Lung | Capmatinib, Cabozantinib |
| | D1010Y, Exon 14 | Cancer | |
| | splice mutation, | | |
| | Y1003C, Y1003F, | | |
| | Y1003N | | |
| MTOR | E2014K, E2419K | Bladder Cancer | Everolimus |
| MTOR | L1460P, L2209V, | Renal Cell Carcinoma | Temsirolimus |
| | L2427Q | | |
| MTOR | Q2223K | Renal Cell Carcinoma | Everolimus |
| NRAS | Oncogenic Mutations | Melanoma | Binimetinib, Binimetinib + Ribociclib |
| NRAS | Oncogenic Mutations | Thyroid Cancer | Radioiodine Uptake |
| | | | Therapy +Selumetinib |
| NTRK | Fusions | All Solid Tumors | Larotrectinib, Entrectinib |
| 1 | | | |
| NTRK | Fusions | All Solid Tumors | Larotrectinib, Entrectinib |
| 2 | | | |
| NTRK | Fusions | All Solid Tumors | Larotrectinib, Entrectinib |
| 3 | | | |
| PIK3C | Oncogenic Mutations | Breast Cancer | Alpelisib + Fulvestrant, |
| A | | | Buparlisib + Fulvestrant, |
| | | | Fulvestrant + Taselisib, Alpelisib, |
| | | | Buparlisib, Copanlisib, GDC-0077, |
| | | | Serabelisib, Taselisib |
| PTCH1 | Truncating Mutations | Embryonal Tumor | Sonidegib |
| PTCH1 | Truncating Mutations | Skin Cancer, Non- | Sonidegib, Vismodegib |
| | | Melanoma | |
| RET | Oncogenic Mutations | Medullary Thyroid Cancer | LOXO-292 |

Level 4
Biological Evidence: 14 Genes, 32 Alterations

| | | | |
|---|---|---|---|
| ALK | L1196M, C1156Y, 11171N, | Non-Small | Lorlatinib |
| | G1269A | Cell Lung | |
| | | Cancer | |
| ATM | Oncogenic Mutations | All Solid | Olaparib |
| | | Tumors | |
| BRAF | L597, D287H, D594, F595L, | All Tumors | PLX8394 |
| | G464, G466, G469, G596, N581, | | |
| | S467L, V459L, K601 | | |
| CDKN2A | Oncogenic Mutations | All Solid | Abemaciclib, |
| | | Tumors | Palbociclib, Ribociclib |
| EGFR | A289V, R108K, T263P, | Glioma | Lapatinib |
| | Amplification | | |
| EGFR | D761Y | Non-Small | Osimertinib |
| | | Cell Lung | |
| | | Cancer | |
| EWSR1 | EWSR1-FLI1 Fusion | Ewing | TK216 |
| | | Sarcoma | |
| FGFR1 | Oncogenic Mutations | All Solid | AZD4547, BGJ398, |
| | | Tumors | Debio1347, |
| | | | Erdafitinib |
| FGFR2 | Oncogenic Mutations | All Solid | AZD4547, BGJ398, |
| | | Tumors | Debio1347, |
| | | | Erdafitinib |
| FGFR3 | Oncogenic Mutations | All Solid | AZD4547, BGJ398, |
| | | Tumors | Debio1347, |
| | | | Erdafitinib |
| KRAS | Oncogenic Mutations | All Tumors | Binimetinib, |
| | | | Cobimetinib, |
| | | | Trametinib |
| MTOR | Oncogenic Mutations | All Solid | Everolimus, |
| | | Tumors | Temsirolimus |
| NF1 | Oncogenic Mutations | All Solid | Cobimetinib, |
| | | Tumors | Trametinib |
| PTEN | Oncogenic Mutations | All Tumors | AZD8186, |
| | | | GSK2636771 |
| SMARCB1 | Oncogenic Mutations | All Tumors | Tazemetostat |

Level R1
Standard care resistance: 4 Genes, 5 Alterations

| | | | |
|---|---|---|---|
| EGFR | Exon 20 insertion, | Non-Small Cell Lung | Afatinib, Erlotinib, |
| | T790M | Cancer | Gefitinib |
| KRAS | Oncogenic Mutations | Colorectal Cancer | Cetuximab, |
| | | | Panitumumab |
| NRAS | Oncogenic Mutations | Colorectal Cancer | Cetuximab, |
| | | | Panitumumab |
| PDGFRA | D842V | Gastrointestinal Stromal | Imatinib |
| | | Tumor | |

Level R2
Clinical evidence of resistance: 4 Genes, 14 Alterations

| | | | |
|---|---|---|---|
| ALK | G1202R, 11171N | Non-Small Cell Lung Cancer | Alectinib |
| ALK | L1196M, C1156Y, | Non-Small Cell Lung Cancer | Crizotinib |
| | G1269A | | |
| EGFR | C7975, C797G | Non-Small Cell Lung Cancer | Osimertinib |
| EGFR | D761Y | Non-Small Cell Lung Cancer | Gefitinib |
| KIT | Exon 17 mutations | Gastrointestinal Stromal | Imatinib, Sunitinib |
| | | Tumor | |
| KIT | T670I, V654A | Gastrointestinal Stromal | Imatinib |
| | | Tumor | |
| MET | Amplification | Non-Small Cell Lung Cancer | Erlotinib, Gefitinib |
| MET | D1228N | Non-Small Cell Lung Cancer | Cabozantinib, Capmatinib, |
| | | | Crizotinib |
| MET | Y1230H | Non-Small Cell Lung Cancer | Crizotinib |

1. A DNA sequence analysis system, comprising:
computing module, configured to:
receive DNA sequencing data;
wherein the DNA sequencing data is a plurality of
non-paired sequenced reads, or
paired sequenced reads with unsequenced DNA between them, of at least one genome of a subject;
receive at least one DNA reference sequence and reference DNA alignment data for the at least one DNA reference sequence;
analyze the reference DNA alignment data and the at least one DNA reference sequence to obtain a plurality of distinct reference mismatch identifying data type outputs, for non-paired reads comprising:
i) an abnormal read depth identifying data type output,
ii) a single nucleotide variant identifying data type output,
iii) a short insertion/deletion (indel) identifying data type output, or
iv) a split-read mapping identifying data type output;
and for paired reads additionally comprising:
v) a discordant mate identifying data type output,
vi) an unmapped mate identifying data type output, or
vii) a discordant read orientation identifying data type output;
evaluate each respective genome position of the at least one genome of the subject using a joint analysis of all distinct data type outputs of the plurality of reference mismatch identifying data type outputs to identify all subject-specific genome variants corresponding to at least one genome variant type of a plurality of genome variant types;
wherein each potential reference genome variant relative to the at least one reference DNA sequence is at least one of:
a) a single-nucleotide variant,
b) a short indel,
c) a deletion,
d) an insertion of a non-reference DNA sequence,
e) an inversion,
f) a duplication,
g) a translocation between separate contiguous DNA stretches, or
h) a change in a copy number of parental alleles;
wherein a speed of jointly identifying all genome variants of the plurality of genome variant types by jointly considering all distinct data type outputs of the plurality of reference mismatch identifying data type outputs is at least 1.5 fold higher than a speed of obtaining the same genome variants of the plurality of genome variant types, by separately identifying and then combining:
i) one or more genome variants of each respective genome variant type of the plurality of genome variant types, or
ii) one or more genome variants of each subset of respective genome variant types of the plurality of genome variant types;
wherein an accuracy of jointly identifying all genome variants of the plurality of genome variant types by jointly considering all distinct data type outputs of the plurality of reference mismatch identifying data type outputs is equal to or higher than an accuracy of separately identifying the same all genome variants of the plurality of genome variant types, by separately identifying:
i) all genome variants of each respective genome variant type of the plurality of genome variant types, or
ii) all genome variants of each subset of respective genome variant types of the plurality of genome variant types.

2. The DNA sequence analysis system of claim 1, wherein a particular subject-specific genome variant is associated with a particular disease or a particular disorder.

3. The DNA sequence analysis system of claim 2, wherein the particular disease or the particular disorder is a cancer.

4. The DNA sequence analysis system of claim 2, wherein the particular subject-specific genome variant associated with the particular disease or the particular disorder corresponds to at least one abnormal genotype difference in at least one diseased body part of the subject from a non-diseased body part of the subject; and
further comprises:
identifying the at least one abnormal genotype difference, by jointly comparing each subject-specific genome variant identified in a first genome of the at least one diseased body part of the subject to each subject-specific genome variant identified in a second genome of the non-diseased body part of the subject.

5. The DNA sequence analysis system of claim 1, wherein, as part of the evaluation of each respective genome position of the at least one genome of the subject using a joint analysis of all distinct data type outputs of the plurality of reference mismatch identifying data type outputs, the computing module is further configured to:
produce during the evaluation at least one breakpoint cluster of reads supporting the same variant type,
wherein a breakpoint cluster at a specific reference genome position is a set of reads or unsequenced DNA between paired reads supporting a breakpoint at that location for a specific variant type of a length approximation compatible with reference mismatch identifying data type outputs obtained from said reads;
identify at least one variant from the plurality of breakpoint cluster of reads by using common statistical evaluation for different variant types,
wherein the identified presence of one variant type affects the evaluation of another variant type.

6. The DNA sequence analysis system of claim 1, wherein, as part of the evaluation of each respective genome position of the at least one genome of the subject using a joint analysis of all distinct data type outputs of the plurality of reference mismatch identifying data type outputs, the computing module is further configured to apply during the evaluation a nucleotide content weighting method for each genome position.

7. The DNA sequence analysis system of claim 1, wherein, as part of the evaluation of each respective genome position of the at least one genome of the subject using a joint analysis of all distinct data type outputs of the plurality of reference mismatch identifying data type outputs, the computing module is further configured to apply during the evaluation a nucleotide content bias normalization for each genome position.

8. The DNA sequence analysis system of claim 1, wherein, as part of the evaluation of each respective genome position of the at least one genome of the subject using a joint analysis of all distinct data type outputs of the plurality of reference mismatch identifying data type outputs, the computing module is further configured to apply during the evaluation a dinucleotide repeat bias normalization for each genome position.

9. The DNA sequence analysis system of claim 5, wherein, as part of the evaluation of each respective genome position of the at least one genome of the subject using a joint analysis of all distinct data type outputs of the plurality of reference mismatch identifying data type outputs, the computing module is further configured to:
utilize during the evaluation at least one sequence window with independently sliding borders for finding copy number changes based on read depth, and
to add at least one window with the copy number change borders to the breakpoint clusters supporting deletion and duplication type variants.

10. A method, comprising:
receiving, by a computing module, DNA sequencing data;
wherein the DNA sequencing data is a plurality of
non-paired sequenced reads, or
paired sequenced reads with unsequenced DNA between them, of at least one genome of a subject;
receiving, by the computing module, at least one DNA reference sequence and reference DNA alignment data for the at least one DNA reference sequence;
analyzing, by computing module, the reference DNA alignment data and the at least one DNA reference sequence to obtain a plurality of distinct reference mismatches;
identifying, by computing module, data type outputs, for non-paired reads comprising:
i) an abnormal read depth identifying data type output,
ii) a single nucleotide variant identifying data type output,
iii) a short insertion/deletion (indel) identifying data type output,
iv) a split-read mapping identifying data type output
and, for paired reads, additionally comprising:
v) a discordant mate identifying data type output,
vi) an unmapped mate identifying data type output,
vii) a discordant read orientation identifying data type output;
evaluating, by computing module, each respective genome position of the at least one genome of the subject using a joint analysis of all distinct data type outputs of the plurality of reference mismatch identifying data type outputs to identify all subject-specific genome variants corresponding to at least one genome variant type of a plurality of genome variant types;
wherein each potential reference genome variant relative to the at least one reference DNA sequence is at least one of:
a) a single-nucleotide variant,
b) a short indel,
c) a deletion,
d) an insertion of a non-reference DNA sequence,
e) an inversion,
f) a duplication,
g) a translocation between separate contiguous DNA stretches,
h) a change in a copy number of parental alleles;
wherein a speed of jointly identifying all genome variants of the plurality of genome variant types by jointly considering all distinct data type outputs of the plurality of reference mismatch identifying data type outputs is at least 1.5 fold higher than a speed of obtaining the same genome variants of the plurality of genome variant types, by separately identifying and then combining:
i) one or more genome variants of each respective genome variant type of the plurality of genome variant types, or
ii) one or more genome variants of each subset of respective genome variant types of the plurality of genome variant types;
wherein an accuracy of jointly identifying all genome variants of the plurality of genome variant types by jointly considering all distinct data type outputs of the plurality of reference mismatch identifying data type outputs is equal to or higher than an accuracy of separately identifying the same all genome variants of the plurality of genome variant types, by separately identifying:
i) all genome variants of each respective genome variant type of the plurality of genome variant types, or
ii) all genome variants of each subset of respective genome variant types of the plurality of genome variant types.

12. The method of claim 11, wherein a particular subject-specific genome variant is associated with a particular disease or a particular disorder.

13. The method of claim 12, wherein the particular disease or the particular disorder is a cancer, wherein a diseased part of a body has a genotype different by one or more breakpoints from a healthy part of the body.

14. The method of claim 13, wherein the particular subject-specific genome variant associated with a cancer is listed in Table 3.

15. The method of claim 10, further comprising
(c) determining, by computer module, if the at least one genome of the subject comprises a particular validated genome variant associated with a cancer, wherein identifying that the at least one genome of the subject comprises the particular validated genome variant associated with the cancer selects the subject for at least one of a monitoring method or a diagnostic method relating to monitoring or diagnosing the cancer; and
(d) performing the at least one of the monitoring method or the diagnostic method relating to monitoring or diagnosing the cancer in the subject identified as having the genome comprising the particular validated genome variant associated with the cancer.

16. The method of claim 15, wherein the monitoring method or the diagnostic method comprises at least one of a blood test, an imaging protocol, a biopsy, or a histopathological analysis.

17. The method of claim 10, further comprising
b) determining, by computer module, if the at least one genome of the subject comprises a particular validated genome variant associated with the cancer,
wherein identifying that the at least one genome of the subject comprises the particular validated genome variant associated with the cancer selects the subject as in need of at least one therapeutic regimen, wherein the therapeutic regimen comprises a protocol for reducing cancer cell number in the subject, wherein the protocol comprises at least one of:
(i) a therapeutic agent used to treat the cancer;
(ii) chemotherapy used to treat the cancer;
(iii) radiation used to treat the cancer; or
(iv) surgical resection of the cancer; and
c) implementing the therapeutic regimen on the subject identified as having the genome comprising the particular validated genome variant associated with the cancer.

18. The method of claim 10, further comprising
(a) obtaining a subject, wherein the subject has a preliminary diagnosis of a cancer, wherein the preliminary diagnosis is based on results from at least one diagnostic method for detecting the cancer in the subject; and
(b) determining, by computer module, if a genome of the subject comprises a particular validated genome variant associated with a cancer,
wherein if the particular validated genome variant associated with the cancer is not detected in the subject's genome, a proposed treatment regimen of the cancer in the subject based on the preliminary diagnosis is not recommended, thereby reducing the frequency of ineffective treatment regimens of the cancer in the subject.

19. The method of claim 10, further comprising
(a) obtaining a subject, wherein the subject has a preliminary diagnosis of a cancer, wherein the preliminary diagnosis is based on results from at least one diagnostic method for detecting the cancer in the subject; and
(b) determining, by computer module, if a genome of the subject comprises a particular validated genome variant associated with a cancer,
wherein if the particular validated genome variant associated with the cancer is not detected in the subject's genome, the preliminary diagnosis of the cancer in the subject is identified as a false positive diagnosis of the cancer in the subject, thereby reducing the frequency of false positive diagnoses of the cancer in the subject.
